(19) United States
(12) Patent Application Publication
(10) Pub. No.: US 2006/0264771 A1
(43) Pub. Date: Nov. 23, 2006

(54) APPARATUS FOR EVALUATING CARDIOVASCULAR FUNCTIONS

(75) Inventors: Kang-Ping Lin, Jhongli City (TW); Hong-Dun Lin, Taipei City (TW); Bor-Iuan Jan, Pingtung City (TW); Mei-Feng Chen, Guantian Township (TW); Chih-Hsiung Yu, Taoyuan City 330 (TW)

Correspondence Address: NIKOLAI & MERSEREAU, P.A., 900 SECOND AVENUE SOUTH, SUITE 820, MINNEAPOLIS, MN, 55402, US

(73) Assignee: DAILYCARE BIOMEDICAL INC., Chung-Li City (TW)

(21) Appl. No.: 11/306,723

(22) Filed: Jan. 9, 2006

(30) Foreign Application Priority Data
May 20, 2005 (TW) 94116790

(51) Int. Cl. A61B 5/04; A61B 5/02

(52) U.S. Cl. 600/513; 600/481

(57) ABSTRACT

An apparatus is provided for evaluating cardiovascular functions. The apparatus combines the principles of measuring pressure and pulse signals, and monitors blood pressure, pulse wave velocity and electrocardiogram (ECG) signals simultaneously. An inflatable cuff measures blood pressure and pulse wave velocity. The apparatus also calculates systolic pressure, diastolic pressure, mean arterial pressure and other parameters related to blood pressure. The apparatus not only calculates vascular Stiffness Index (SI), vascular Reflection Index (RI) and other arterial related parameters but also Pulse Wave Velocity (PWV), heartbeat, QRS interval, ST segment, and other ECG parameters.

### BACKGROUND OF THE INVENTION

1. Field of the Invention

The present invention relates to an apparatus for monitoring physiology signals, more particularly to a device for monitoring blood pressure signals, pulse signals and electrocardiogram (ECG) signals and further for acquiring blood pressure parameters, blood vessel parameters and parameters related to ECG signals for estimating cardiac and vascular conditions.

2. Description of Prior Arts

High blood pressure, heart disease, hardening of the arteries, myocardial infarction, stroke and relative diseases of the cardiovascular system have threatened the health of humans for a long time. More particularly, aging and related diseases of the cardiovascular system are of huge concern, particularly as the average human life-span continues to lengthen.

From the view of medical treatment, conditions, such as high blood pressure, hardening of the arteries, myocardial infarction, heart disease and stroke etc., are related to disease of the cardiovascular system. Appropriate diet and exercise are considered important in protection of the cardiovascular system, but monitoring the heart, blood pressure and blood vessels provides direct and immediate awareness of such medical situations.

One of the most simple and straightforward methods for monitoring cardiac conditions is electrocardiogram(ECG) record. Electrical conduction of the whole heart muscle is recorded by cardiograph and presented by electrocardiogram. Clinically, electrocardiograms are usually applied in cardiac diagnosis.

Many types of blood pressure monitors, such as mercury sphygmomanometer, wrist-type or arm-type, are available and easily used. According to the World Health Organization's (WHO), systolic pressure is defined below 120 mmHg and diastolic pressure is below 80 mmHg, standards for monitoring cardiovascular diseases.

Clinical, non-invasive evaluation of blood vessels, such as ultrasonic equipment, is used to diagnose peripheral vascular diseases. Additionally, pressure principle can be used in blood vessel evaluation, by measuring blood pressure in the ankle and upper arm to estimate vascular obstructions. Further, the method of monitoring pulse wave in co-operation with electrocardiogram is used to calculate the Pulse Wave Velocity (PWV) for evaluating hardening of the arteries. For example, No. VP-1000 non-invasive vascular screening device produced by the Colin Corporation estimates both vascular obstruction and calculates PWV. The above-mentioned methods are widely applied to estimate the health of the heart and blood vessels. 12 lead ECG analyses are used frequently in clinical environments, but are not convenient for home use, such that more convenient blood pressure monitors are used.

Evaluation of heart, blood pressure and blood vessel conditions at the same time is achieved using a plurality of devices which is not effective in terms of time, money or effort, and is further in convenient. Thus, for efficient acquisition of parameters to provide instant signals for understanding the conditions of the health, the principles of detecting pressure and monitoring electric signals are integrated into a cardiovascular device for evaluating blood pressure signals, pulse wave signals and ECG signals at the same time. Not only blood pressure and pulse wave are measured through an inflatable cuff but also blood pressure parameters, such as systolic pressure, diastolic pressure and mean arterial pressure, and vascular parameters, such as vascular Stiffness Index (SI) and vascular Reflection Index (RI), and acquisition of ECG signals with synchronous cooperation for further calculating ECG parameters, such as Pulse Wave Velocity (PWV), heartbeats, QRS interval, and ST segment interval.

### SUMMARY OF THE INVENTION

Accordingly, the primary object of the present invention is to provide an apparatus for evaluating cardiovascular functions. The apparatus monitors electrocardiogram signals, blood pressure signals and pulse wave signals at the same time, deriving ECG parameters to provide complete information regarding cardiac and vascular conditions.

Another object of the present invention is to provide various objects and advantages. The apparatus of the present invention will be more readily understood from the following detailed descriptions when read in conjunction with the appended drawings. The apparatus combines pressure and electrical signal measurement, and monitors blood pressure, pulse wave velocity, and electrocardiogram signals simultaneously.

The present invention provides an apparatus for monitoring blood pressure and vascular condition at the same time. The apparatus monitors blood pressure while implementing a procedure for regulating pressure through an inflatable cuff to acquire a pulse signal for evaluating vascular aging.

The present invention also provides an integrated device, measuring physiology signals, which combines monitoring of blood pressure signals, pulse wave signals and ECG signals. Multiple signals are thus acquired and conveniently, provide multiple parameters, widely applied in health maintenance and disease prevention.

To achieve the above mentioned objects, the present invention measures blood pressure by combining pulse wave and acquisition ECG signals, providing a device measuring blood pressure, pulse wave, and ECG signals.

The apparatus includes an inflatable cuff wound around a part of testee's body, such as arms, wrist and fingers, a pressure sensor connected to the inflatable cuff for monitoring pressure variation, a CPU controlling the inflatable cuff and activating a pressure procedure and a regulating procedure by a pressure sensor to detect resonance signals during the pressure procedure and recording a pulse signal during the pressure regulation, and at least two sensing electrodes acquiring ECG parameters during the pressure regulation.

Processing the resonance, pulse, and ECG signals using a signal processor module, signals are converted to digital format and delivered to the CPU for analysis, acquiring blood pressure, comprising systolic pressure, diastolic pressure, and mean arterial pressure, and ECG signals, such as QRS interval, ST segment etc., blood parameters, heartbeat, QRS interval, ST segment etc., to provide information regarding heart and vascular conditions.

The objects, features, and effects of the present invention will be more readily understood from the following detailed description of preferred embodiments with the appended drawings.

### BRIEF DESCRIPTION OF THE DRAWINGS

FIG. 1A is a schematic diagram of an inflatable cuff applied on a testee's arm, in accordance with the present invention;

FIG. 1B is a schematic diagram showing compression applied by the inflatable cuff;

FIG. 2 is a schematic diagram showing calculation of blood parameters;

FIG. 3 is a schematic diagram showing pulse waveform of the blood vessel;

FIG. 4 is a schematic diagram showing the relation between pulse waveform and electrocardiogram (ECG) signal;

FIG. 5 is a block schematic diagram of the apparatus according to the present invention;

FIG. 6 is schematic diagram of signal monitoring based on the apparatus of the present invention; and

FIG. 7 is another schematic diagram of signal monitoring based on the apparatus of the present invention.

### DETAILED DESCRIPTION OF THE INVENTION

In the following detailed description of the preferred embodiments, it is to be understood that other embodiments may be utilized and structural changes may be made without departing from the scope the present invention. The invention will be further described in conjunction with the accompanying drawings, which illustrate preferred (best mode) embodiments.

A method and apparatus for evaluating cardiovascular functions is disclosed according to the present invention. The present invention is designed on the basis of sensing pressure theory, and is applied in conventional sphygmomanometers combined with techniques monitoring electrocardiogram. Thus, the invention comprises not only monitors blood pressure and electrocardiogram (ECG) signals, but also various pulse signals through a control pressure method, utilizing unique sequential pressure control of reduction and regulation of pressure. The present invention not only analyzes artery Stiffness Index (SI) and Reflection Index (RI) cooperatively to acquire ECG signals but calculates pulse wave velocity (PWV).

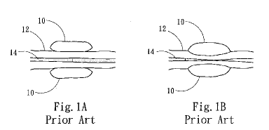

Principle of sphygmomanometric measurement is described as follows for completely understanding of the invention. Non-intrusive sphygmomanometers generally include diagnostic sphygmomanometers, such as mercury sphygmomanometers, and resonance sphygmomanometers applied in most electronic sphygmomanometers. The principles of both two sphygmomanometers are related to restricted blood flow caused by compressing blood vessels through an inflatable cuff and examining blood pressure, such as systolic pressure, diastolic pressure, and mean arterial pressure, through pressure variations in the inflatable cuff. For resonance sphygmomanometers, an inflatable cuff 10 is applied to a testee's arm 12, as shown in FIG. 1A, and pressure raised by inflating beyond systolic pressure. Arterial blood vessels of the testee's arm are compressed and obstructed as shown in FIG. 1B. As the pressure of the inflatable cuff 10 is gradually decreased, blood flow resumes through the blocked blood vessel 14, producing a slight pulse and pressure oscillation in the inflatable cuff is produced by the systolic variation of blood flow. Signals of pressure variation within the inflatable cuff 10 and oscillating pressure signals reflected by blood vessel 14 are detected by a pressure sensor.

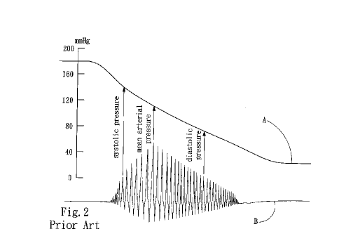

Referring to FIG. 2, two signals are separated after processing, with curve A of pressure variation in inflatable cuff 10 and the curve B of oscillating pressure reflected by blood vessel are acquired. When the maximum value of the oscillating amplitude occurs, the pressure is set as mean arterial pressure (MAP), and the systolic pressure and the diastolic pressure are estimated with mean arterial pressure by rule of thumb. For example, if the pressure of the inflatable cuff 10 changes from high to low, diastolic pressure occurs at the particular position where half of the maximum amplitude occurs. However, diastolic pressure occurs at 0.78 multiple value of the maximum amplitude. The value for estimation is adjusted and corrected in accordance with different elements used in device and clinical results.

Vascular pulse monitoring can utilize an optical method based on measuring various absorption and reflection quantities caused by various concentration of oxygen contained in blood, and a pressure method utilizes opposite pressure caused by blood flow, which changes with the contraction and diastole of the heart, especially when the blood vessel is compressed.

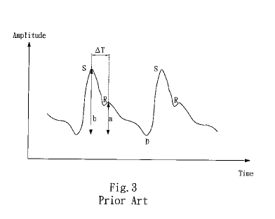

For example, if a pulse wave is obtained as shown in FIG. 3, the first pulse wave peak S occurs by opposite pressure produced by heart contraction, and pulse wave valley D by opposite pressure produced by heart diastole.

The second pulse wave peak R is a reflected wave signal transmitted along the clavicle artery to lower limbs. The delay time between the first wave pulse peak S and the second pulse wave peak R is determined by movement of the back-and-forth pulse along the path from the clavicle artery to lower limbs, the conduction time being proportional to the height and affected by age and elasticity of the blood vessel. Better vascular elasticity provides better pulse absorbency, so conduction time is longer. Vascular indurations can be approximately calculated using the following formula:

$$SI = \frac{Subject height}{Δ T} in ms \qquad ( 1 )$$

SI: vascular induration index

Subject Height: testee's height;

ΔT: delay time between two peaks;

Height difference between two peaks can also be used to evaluate arterial constriction. The symbol is indicated as RI, and the formula is

$$RI = \frac{a}{b} \times 100 \% \qquad ( 2 )$$

RI: vascular elastic index;

a: amplitude of the first wave peak R;

b: amplitude of the second wave peak S

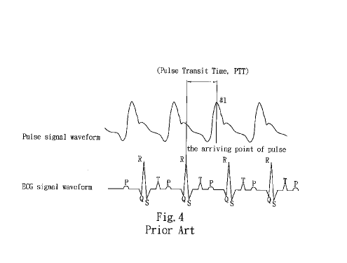

Pulse Wave Velocity (PWV) is calculated using ECG signals and pulse wave signals. PWV is mainly used in evaluation velocity of a pulse produced by blood pumped from the heart to extremities. PWV increases indicate increased vascular wall rigidity. Hence, the PWV relates to above-mentioned artery Stiffness Index (SI). If a blood vessel becomes excessively rigid, the conductive pulse wave is not easily absorbed by the vascular walls, so Pulse Wave Velocity increases. Blood vessels showing relatively higher elasticity allow the conductive pulse wave to be more easily absorbed by the vascular walls, and Pulse Wave Velocity decreases. Pulse Wave Velocity is related to occurrence of cardiovascular disease. Increased PWV value indicates increased probability of arteriosclerosis. Referring to FIG. 4, Pulse Wave Velocity is calculated from the following formula:

$$PWV = \frac{D}{PTT} \qquad ( 4 )$$

PWV: Pulse Wave Velocity

D: conduction distance of Pulse Wave

PTT: Pulse Transit Time

PWV is acquired by transit time referring to a reference time and ECG signals. Because the R wave of the ECG signals is easily acquired, the R wave is usually set as a reference basis and the first wave peak of the pulse wave is set as the arrival point. Within conduction distance D, the arrival point changes with testee's height and the position of pulse wave acquisition.

The foregoing is the principles of operating blood pressure parameters and relative vascular parameters. Because the sphygmomanometer on the market has no vascular pulses analysis function and monitoring ECG signals function, it is inconvenient for using by the public.

The apparatus of the present invention overcomes the limited monitoring of blood pressure signals, ECG signals, and pulse signals by combing monitoring of electronic signals and pressure measurement, providing complete evaluation of cardiovascular conditions and improving home use. The embodiment of the apparatus is described as follows.

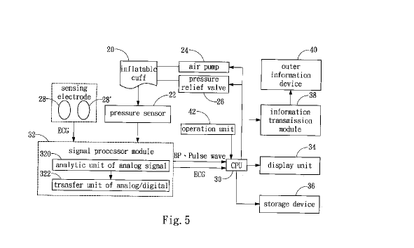

FIG. 5. is a block schematic diagram of an apparatus of the present invention, comprising an inflatable cuff 20 around a part of the testee's body, such as the upper arm, wrist, or finger, a pressure sensor 22 connected to the inflatable cuff 20 monitoring pressure variations in the inflatable cuff 20, an air pump 24 and a pressure relief valve 26 together connected to the inflatable cuff 20, adjusting the inner pressure thereof, a CPU 30 acting as an inner-control operation unit, controlling a pressure procedure, a regulation pressure procedure, and monitoring a resonance signal and a pulse signal, at least two sensing electrodes 28 and 28′ contacting with the testee's skin, synchronously acquiring ECG signals during the regulation pressure procedure, a signal processor module 32 processing the resonance signal and pulse signal detected by the pressure sensor 22 and the ECG signals detected by the sensing electrodes 28 and 28′, including an analytic unit for an analog signal 320 separating and amplifying signals and filtering waves, and a analog/digital converter 322 delivering converted signals to the CPU 30.

After receiving resonance signals, the pulse wave signals, and ECG signals, the CPU 30 operates predetermined blood pressure parameters, ECG parameters, and pulse parameters through inner programs to acquire blood pressure parameters, such as systolic pressure, diastolic pressure, and mean arterial pressure, ECG parameters, such as heartbeat, QRS interval, and ST segment, and vascular parameters, such as artery Stiffness Index (SI), artery Reflection Index (RI), and Pulse Wave Velocity (PWV), and then delivers the acquired parameters to a display unit 34, such as a liquid crystal display or LED.

The present invention also comprises a storage device 36, such as RAM, ROM, EEPROM, Flash, RAM . . . etc., storing signals and parameters received and operated by the CPU 30. Through an information transmission module 38, such as USB, RS232, Blue Tooth, or IR interface and modem, signals and parameters stored in the storage unit 36 are delivered to an outer information device 40, such as a computer, PDA, cell phone, database server . . . etc., to provide health management.

The present invention comprises an operation unit 42 electrically connected to the CPU 30, further controlling the device, selecting the monitor function and additional functions, such as input of patient information and date, adding, deleting, and delivering the information stored in the storage unit 36, etc. The type of operation unit 42 can be a pressing-button, a knob, or a touch plate . . . etc.

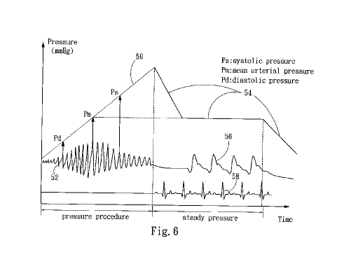

FIG. 6 shows a schematic diagram of signals measurement, according to the present invention. The monitoring principle of the present invention is based on the principle of sensing pressure and sensing electric signals. Therein, in regards to sensing pressure, it is further divided into sensing blood pressure and sensing pulse wave. The inflatable cuff 20 is wound around a part of the testee's body, such arms, wrist, and finger etc. The apparatus further includes sensing electrodes 28 and 28′ contacting with the testee's skin, such the skin of the testee's left hand and right hand to record Lead I ECG signals, or the testee's right hand and left leg to record Lead II ECG signals.

First, in regards to sensing pressure, the CPU 30 controls the inflatable cuff 20 by inflation, as in the rising pressure curve 50 shown. Pressure is raised until a predetermined value, such as 180 mmHg, and then maintained. During the raising process, resonance signals reflected by the vascular wall are acquired simultaneously by the pressure sensor 22. The pressure variation signals detected by the pressure sensor 22 are delivered to the analytic unit of analog signal 320 and separated. The acquired resonance signals 52 are shown. After analog/digital converted, the maximum value of the resonant signals 52 is calculated by the CPU 30, and signals are compared to the rising pressure curve 50, the pressure at the position of the maximum amplitude defined as mean arterial pressure Pm.

Maximum amplitude is multiplied with finite values, such 0.5 or 0.8, and respectively adjusted according to the characteristic of elements or clinical results to determine values and positions of the systolic pressure Ps and diastolic pressure Pd corresponding to the rising pressure curve 50. Systolic pressure Ps and diastolic pressure Pd are thus acquired. During pressurization, the pressure of the inflatable cuff is adjusted according to a predetermined stationary pressure value, such as 110 mmHg, 100 mmHg, or 90 mmHg etc., or one of the forgoing acquired systolic pressure Ps, mean arterial pressure Pm, and diastolic pressure Pd. The predetermined value shown in FIG. 6 is mean arterial pressure. Pressure relief valve 26 is controlled through the CPU 30 to slightly decrease the pressure of the inflatable cuff 20, with predetermined value maintained for a predetermined time.

Pressure variation of the inflatable cuff 20 is shown as pressure curve 54, and through the pressure sensor 22 maintained pressure is needed to record at least one complete pulse wave, and synchronously record the pulse signal 56 of the pressure reflected by vascular wall. On the other side, the sensing electrode 28 and 28′ detects ECG signal 58. Pulse signals 56 and ECG signals 58 are processed in the analog format through signal processor module 32, such as amplifying and filtering . . . etc., and converted to digital format signals and delivered to the CPU 30 for calculating ECG parameters and vascular parameters, such as heartbeat, QRS interval, ST segment . . . etc, and vascular parameters, such as artery Stiffness Index (SI), artery Reflection Index artery Reflection Index (RI), and Pulse Wave Velocity (PWV). Thus, the information of both heart and blood vessels is provided and referred, providing evaluation of cardiovascular conditions.

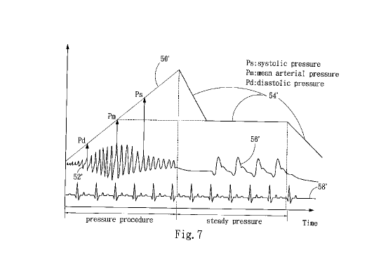

In reference to ECG recording, the sensing electrodes 28 and 28′ are switched on during pressure procedure to monitor electrocardiogram (ECG) signals. FIG. 7 is another schematic diagram showing signals monitoring based on the apparatus of the present invention. The signals are not only used to calculate Pulse Wave Velocity (PWV) but also provide preferred reliability of ECC parameters through recording over a long period of ECG signals 58′. The present invention is not only used to acquire blood pressure signals but also has the functions of calculating blood pressure parameters and relative ECG parameters through pressure control method proceeded and sensing electrodes to acquire blood pressure signals, pulse signals and ECG waveform at the same time. Hence, not only the relative cardiovascular parameters be acquired conveniently but also risk factors of heart, blood vessels, and blood pressure can be monitored. Thus, the apparatus of the present invention is particularly helpful to prevent disease and protect health.

The present invention is thus described. It will be obvious that the same may be varied in many ways. Such variations are not to be regarded as a departure from the sprit and scope of the present invention, and all such modifications as would be obvious to one skilled in the art are intended to be included within the scope of the following claims.

What is claimed is:

1. An apparatus for evaluating cardiovascular functions, comprising:
an inflatable cuff applied to a testee, connected to a pressure sensor;
a CPU controlling the inflatable cuff via the pressure sensor to perform a pressure procedure and a regulation procedure, detecting a resonance signal and a pulse signal during the pressure procedure and the regulation procedure;
at least two sensing electrodes contacting with a skin of the testee, acquiring electrocardiogram (ECG) signals; and
a signal processor module receiving the resonance signal, the pulse signal, and the ECG signals, converting the resonance, pulse and ECG signals into a digital format and delivering to the CPU for operation of physiological signals to acquire at least one blood pressure parameter, at least one blood vessel parameter, and at least one ECG parameter.

2. The apparatus for evaluating cardiovascular functions as recited in claim 1, further comprising a display unit electrically connected to the CPU displaying the at least one blood pressure parameter, the at least one blood vessel parameter, and the at least one ECG parameter.

3. The apparatus for evaluating cardiovascular functions as recited in claim 1, wherein the inflatable cuff is applied to an arm, a wrist, or a finger of the testee.

4. The apparatus for evaluating cardiovascular functions as recited in claim 1, wherein the signal processor module comprises:
an analog signal analytic unit processing the resonance, pulse and ECG signals; and
a transfer unit converting the processed signals to digital format.

5. The apparatus for evaluating cardiovascular functions as recited in claim 1, further comprising a pressure pump and a pressure relief valve connected to the inflatable cuff, the pressure of the inflatable cuff adjusted by the pressure pump and the pressure relief valve.

6. The apparatus for evaluating cardiovascular functions as recited in claim 1, wherein the at least one blood pressure parameter comprises at least one of systolic pressure, mean arterial pressure, and diastolic pressure.

7. The apparatus for evaluating cardiovascular functions as recited in claim 1, wherein the at least one blood vessel parameter comprises at least one of artery Stiffness Index (SI), artery Reflection Index (RI), and Pulse Wave Velocity (PWV).

8. The apparatus for evaluating cardiovascular functions as recited in claim 1, wherein the at least one electrocardiogram (ECG) parameter comprises at least one of heartbeat, QRS interval, and ST segment.

9. The apparatus for evaluating cardiovascular functions as recited in claim 1, wherein a pressure applied to the inflatable cuff increases to at least 140 mmHg.

10. The apparatus for evaluating cardiovascular functions as recited in claim 1, wherein the inflatable cuff is controlled by the CPU to maintain the pressure of the inflatable cuff at a predetermined pressure for a predetermined time.

11. The apparatus for evaluating cardiovascular functions as recited in claim 10, wherein the predetermined value is determined according to the at least one blood pressure parameter acquired in the pressurizing procedure.

12. The apparatus for evaluating cardiovascular functions as recited in claim 10, wherein the predetermined time is needed to detect at least one complete pulse waveform.

13. The apparatus for evaluating cardiovascular functions as recited in claim 1, further comprising a storage unit connected to the CPU, storing the at least one electrocardiogram (ECG) signal, pulse signal, blood pressure parameter, blood vessel parameter, and electrocardiogram (ECG) parameter.

14. The apparatus for evaluating cardiovascular functions as recited in claim 13, wherein the storage unit is ROM, RAM, or EEPROM.

15. The apparatus for evaluating cardiovascular functions as recited in claim 13, further comprising an information transmission module connected to the CPU to deliver the at least one electrocardiogram (ECG) signal, pulse signal, blood pressure parameter, blood vessel parameter, and electrocardiogram (ECG) parameter stored in the storage device to an external information device.

16. The apparatus for evaluating cardiovascular functions as recited in claim 15, wherein the information transmission module is a USB transmission interface, a RS232 transmission interface, a Blue Tooth transmission interface, or a IR interface and modem.

17. The apparatus for evaluating cardiovascular functions as recited in claim 15, wherein the external information device is a computer, a digital personal assistant, a cell phone, or a database server.

18. The apparatus for evaluating cardiovascular functions as recited in claim 1, further comprising a control unit electrically connected to the CPU to further control the actions of the whole apparatus by setting the control unit.
